(19) United States
(12) Patent Application Publication
(10) Pub. No.: US 2024/0366780 A1
(43) Pub. Date: Nov. 7, 2024

(54) PREPARATORY PROCESS

(71) Applicant: TELIX PHARMACEUTICALS (INNOVATIONS) PTY LTD, Victoria (AU)

(72) Inventors: Oliver William STERRITT, North Melbourne (AU); Michael Paul WHEATCROFT, North Melbourne (AU)

(21) Appl. No.: 18/687,283

(22) PCT Filed: Sep. 1, 2022
(86) PCT No.: PCT/AU2022/051071

§ 371 Date: Feb. 27, 2024

(30) Foreign Application Priority Data
Sep. 1, 2021 (AU) 2021902837

(51) Int. Cl. A61K 47/68

(52) U.S. Cl. CPC A61K 47/6887; A61K 47/6803; A61K 47/6871; A61K 47/6889

(57) ABSTRACT

The disclosure provides a process of preparing an iron complexed conjugate between a desferrioxamine chelating ligand and a protein, the process being undertaken at a substantially neutral pH. A process of exposing an iron complexed conjugate to a free chelating ligand selected from desferrioxamine, a desferrioxamine analogue, and HBED, at a substantially neutral pH, in order to remove chelated iron from the iron complexed conjugate is also disclosed.

### CROSS-REFERENCE TO RELATED APPLICATION(S)

This application claims priority to Australian provisional application no. 2021902837 (filed on 1 Sep. 2021), the entire contents of which are incorporated herein by reference.

### FIELD OF THE INVENTION

The invention relates to a process for removing iron from a conjugate of a protein and desferrioxamine (DFO) or an analogue thereof.

### BACKGROUND OF THE INVENTION

Protein conjugates with a chelating ligand are of continuing interest, for example, for their potential use as a therapeutic, diagnostic or theranostic agent.

Chelating ligands are typically multi-dentate and are preferably selective for a nuclide of therapeutic or diagnostic potential.

One problem arising in the preparation of protein conjugates particularly at commercial scale is the formation of aggregates due to the relatively harsh synthetic conditions required for various preparation steps. Protein aggregates may be solid aggregates or soluble aggregates.

Aggregate mitigation strategies include adapting the preparation procedures for the conjugates to reduce aggregate formation or purification procedures to separate aggregated protein from the protein conjugates.

There is therefore a continuing need to provide at least alternative processes for preparing protein conjugates with chelating ligands that can provide the desired conjugates in meaningful yields with low levels of aggregate.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the invention provides a process for preparing a conjugate comprising a protein linked with a DFO chelating ligand, comprising exposing an iron chelated conjugate with a free chelating ligand other than EDTA at ambient temperature and neutral pH. In some embodiments, the free chelating ligand is selected from desferrioxamine, a desferrioxamine analogue and HBED, or a combination thereof.

Chelating ligands necessarily include functional groups capable of forming stable complexes with metals. During conjugate synthesis, the coordination sites on the chelating ligand may conveniently be protected by forming a complex with an otherwise inert metal. For example, DFO may be complexed with iron during preparation of a DFO-protein conjugate. The protecting metal must be removed prior to formation of the final therapeutic, diagnostic or theranostic agent, particularly for radionuclides which may have relatively short half-lives.

In another aspect, the invention provides a process for preparing a conjugate of a desferrioxamine chelating ligand linked with a protein, the process comprising combining, at substantially neutral pH, a first composition comprising an iron chelated desferrioxamine chelating ligand and a polar organic solvent and a second composition comprising a protein, to thereby form an iron complexed conjugate of the desferrioxamine chelating ligand complexed to iron linked with the protein.

In a further aspect, the invention provides a process of preparing a conjugate of a desferrioxamine chelating ligand linked with a protein, the process comprising:
conjugating an iron chelated desferrioxamine chelating ligand with a protein to provide an iron complexed conjugate of the desferrioxamine chelating ligand complexed to iron linked with the protein; and
exposing the iron complexed conjugate comprising the desferrioxamine chelating ligand complexed to iron linked with the protein, to a free chelating ligand selected from desferrioxamine, a desferrioxamine analogue and HBED, or a combination thereof, at substantially neutral pH to thereby remove iron from the desferrioxamine chelating ligand of the iron chelated conjugate and provide the conjugate of the desferrioxamine chelating ligand linked with the protein that is substantially free of chelated iron

Another aspect provides a protein conjugate produced by a process of the invention.

### Selected Definitions

Chemical terms are intended to have their general meaning.

The term “alkyl” is intended to include saturated straight chain and branched chain hydrocarbon groups. In some embodiments, alkyl groups have from 1 to 12, 1 to 10, 1 to 8, 1 to 6, or from 1 to 4 carbon atoms. In some embodiments, alkyl groups have from 5-21, from 9-21, or from 11-21 carbon atoms, such as from 11, 13, 15, 17, or 19 carbon atoms. Examples of straight chain alkyl groups include, but are not limited to, methyl, ethyl, n-propyl, n-butyl, n-pentyl, n-hexyl, n-heptyl, and n-octyl. Examples of branched alkyl groups include, but are not limited to, isopropyl, iso-butyl, sec-butyl, tert-butyl, neopentyl, isopentyl, and 2,2-dimethylpropyl.

The term “halo” is intended to include chloro (—Cl), bromo (—Br), fluoro (—F) and iodo (—I) groups. In some embodiments, halo may be selected from chloro, bromo and fluoro, preferably fluoro.

As used herein, the term ‘theranostic’ refers to the ability of compounds/materials to be used for diagnosis as well as for therapy. The term “theranostic reagent” relates to any reagent which is both suitable for detection, diagnostic and/or the treatment of a disease or condition of a patient. The aim of theranostic compounds/materials is to overcome undesirable differences in biodistribution and selectivity, which can exist between distinct diagnostic and therapeutic agents.

As used herein, the term “and/or” means “and”, or “or”, or both.

The term “(s)” following a noun contemplates the singular and plural form, or both.

As used herein, except where the context requires otherwise, the term “comprise” and variations of the term, such as “comprising”, “comprises” and “comprised”, are not intended to exclude further additives, components, integers or steps.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9, and 10) and also any range of rational numbers within that range (for example, 2 to 8, 1.5 to 5.5, and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

Various features of the invention are described with reference to a certain value, or range of values. These values are intended to relate to the results of the various appropriate measurement techniques, and therefore should be interpreted as including a margin of error inherent in any particular measurement technique. Some of the values referred to herein are denoted by the term “about” to at least in part account for this variability. The term “about”, when used to describe a value, may mean an amount within ±10%, ±5%, ±1% or ±0.1% of that value.

Unless context dictates otherwise, any embodiment may be combined with any other embodiment described herein.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1. A graph of percentage high molecular weight (% HMW) aggregates relative to monomer determined by size exclusion chromatography-high performance liquid chromatography (SEC-HPLC) across various steps of the processes described herein for DFO transchelation conditions described in Example 3.

### DETAILED DESCRIPTION OF THE EMBODIMENTS

The invention relates to processes for preparing a conjugate of a DFO chelating ligand linked with a protein. The conjugates described here may conveniently be prepared by conjugating a DFO chelating ligand with a protein. The DFO chelating ligand is typically complexed with iron, which serves as a protecting group for the DFO chelator. Following conjugation, the iron protecting group may be removed by transchelation. Each of these steps may be carried out separately, or in preferred embodiments these steps may be carried out in sequence generally from commercially available starting materials.

The processes described herein include either or both of the improved conjugation and/or transchelation steps described herein. It will be understood that a conjugate produced by the improved conjugation step only can be further processed by any means known in the art (including EDTA mediated transchelation). Further, conjugates prepared by any means known in the art may be subjected to any of the transchelation conditions described herein. It will also be appreciated that a process may include both conjugation and transchelation steps as described herein.

### Conjugation

In one aspect, the processes comprise a step of forming the conjugate comprising conjugating (or coupling) a desferrioxamine chelating ligand complexed to iron with a protein. This step may be carried out by any known conjugation techniques known in the art.

Conveniently, the conjugating step may be carried out at substantially neutral pH. Prior to this invention it was believed that increased pH was preferable for the conjugating step; however, the inventors surprisingly found that the coupling reaction proceeded under substantially neutral pHs. The substantially neutral pH is advantageous as some protein coupling partners may degrade under high pHs (eg pH>8). These protein coupling partners may degrade through denaturation or by forming undesirable high molecular weight aggregates. In addition, in aspects and embodiments of the processes described herein including both conjugation and transchelation steps, the ability to maintain substantially the same pH conditions across both steps reduces the overall time of the production process as it avoids time consuming titrations between steps.

The conjugation step is advantageously carried out at substantially neutral pH. The inventors have shown (see Examples) that the conjugation reaction proceeds at substantially neutral pH in at least comparable yield to the previously believed essential higher pH conditions (see Example 1 which compares pH 7.2, 8.4 and 9.6). During this step, acceptable conjugate to protein ratios are achieved and the process avoids aggregate formation. In some embodiments, the pH of the conjugation step is substantially the same as for the transchelation step. Accordingly, in some embodiments, the pH is from about 7 to about 8. In some embodiments, the pH is physiological pH, for example about 7.4. In some embodiments, the pH is about 7.2. As the conjugate comprises a protein which may already have adopted a tertiary structure, maintaining substantially neutral pH (and/or physiological pH) is advantageous to ensure that the conjugate forms on a solvent exposed position of the protein's tertiary structure and reduces the likelihood of interfering with the protein's intended function.

The iron chelated desferrioxamine chelating ligand may be linked with the protein directly, or these moieties may be linked through a linking group. The linking group may be any suitable linking group, including any of those described herein.

The conjugating step may be carried out in any suitable solvent, typically the protein is provided in an aqueous carrier, preferably an aqueous buffer, and the DFO-chelating ligand may be provided as a composition comprising a polar organic solvent.

Accordingly, typically the second composition may further comprise water, preferably further comprising an aqueous buffer. Any compatible aqueous buffer may be used. Suitable buffers include phosphate buffered saline (PBS). In some embodiments, the second composition is substantially free of organic solvent prior to combining with the first composition.

The first composition may comprise any polar organic solvent that is miscible with water, such as acetonitrile (ACN), dimethylsulfoxide (DMSO) or a combination thereof.

In some embodiments, the second composition (eg comprising the protein, such as an antibody) may be added to the first composition (eg comprising the iron-chelated DFO moiety). However, typically, the first composition is added to the second composition. The first and second composition may be combined by any suitable means and at any suitable rate.

In these embodiments, the minimum concentration of iron complexed DFO chelating ligand in the first composition may be from about 1.25 mg/ml, 1.5 mg/ml, 2 mg/ml, 2.5 mg/ml, 3 mg/ml, 4 mg/ml or 5 mg/ml. The maximum concentration of chelating ligand may be not more than about 10 mg/ml, 7.5 mg/ml or 5 mg/ml. In some embodiments, the concentration of chelating ligand in the first composition comprising solvent may be from about 1.25 mg/ml to about 10 mg/ml or 1.5 mg/ml to about 5 mg/ml. As the second composition typically is aqueous higher concentrations of DFO chelating ligand in the first composition are preferred to minimise the addition of polar organic solvent, which may impact protein stability.

The desferrioxamine chelating ligand is conveniently provided as a complex with iron. The iron may be iron (II) or iron (III). Typically, the iron is chelated to the desferrioxamine chelating ligand.

The desferrioxamine chelating ligand prior to conjugation with the protein may be functionalised with an activating group or a group capable of forming an activating group for the conjugation reaction. For example, the desferrioxamine chelating ligand may comprise a carbonyl moiety activated in the form of a carboxylic acid, an acid halide (eg an acid chloride), a mixed anhydride, or an activated ester (e.g. a tetrafluorophenyl ester. Typically the conjugation step involves forming an amide or ester bond with a side chain of an amino acid residue of the protein.

The desferrioxamine chelating ligand prior to conjugation with the protein may comprise a moiety corresponding to a desired linking group. For example, in some embodiments, the DFO chelating ligand comprises a succinyl moiety, bound at one end to DFO and the other functionalised with an activating group or a group capable of forming an activating group for the conjugation reaction.

In some embodiments, the linking group may be divided with a first portion bound to the desferrioxamine chelating ligand complexed with iron and a second portion bound to the protein. In these embodiments, the conjugating step comprises coupling the first and second portions to form the linking group and hence the conjugate.

In some embodiments, the protein is functionalised with the linking group, which is then conjugated with the desferrioxamine chelating ligand complexed with iron.

In some embodiments, the protein, linking group and desferrioxamine chelating ligand complexed with iron are conjugated in a 3-component coupling, where bonds are formed between the protein and the linking group and the linking group and the desferrioxamine chelating ligand complexed with iron.

Typically, in the conjugating step, the desferrioxamine chelating ligand is provided in a molar excess relative to the protein. For example, the desferrioxamine chelating ligand may be provided in a minimum excess of at least about 1.1:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 4:1, 5:1, 6:1, 7:1, 7.5:1, 8:1, 9:1, 10:1, 11:1 or 12:1 relative to the protein. The maximum molar excess of the desferrioxamine chelating ligand relative to the protein may be not more than about 100:1, 90:1, 80:1, 70:1, 60:1, 50:1, 40:1, 30:1, 20:1, 15:1, 12.5:1, 10:1, 8:1, 7.5:1, 7:1, 6.5:1, 6:1, or 5:1. The molar ratio of desferrioxamine chelating ligand to protein during the conjugating step may be from any of these minimum values to any maximum value provided the minimum is less than the maximum, for example, from about 1.1:1 to about 100:1 or about 1.5:1 to about 15:1, about 2:1 to about 8:1, or about 1.1:1 to about 5:1.

In some embodiments, the process does not comprise a purification step following conjugation, and the iron complexed conjugate is provided to the exposing step described herein in the presence of unreacted desferrioxamine chelating ligand complexed to iron and optionally functionalised with an activating group or a group capable of forming an activating group for the conjugation reaction.

### Transchelation

In one aspect, the processes described herein comprise exposing an iron complexed form of the conjugate to a free chelating ligand at substantially neutral pH. The iron complexed conjugate comprises iron chelation to the DFO chelating ligand. The free chelating ligand may be any iron chelator other than EDTA.

DFO and its analogues (including DFO*, DFOsq, DFONCS, DFO*sq, and DFO*NCS) are selective chelating ligands for desired nuclides of therapeutic, diagnostic and/or theranostic potential. In particular, DFO and its analogues are selective chelators for ⁸⁹Zr. ⁸⁹Zr is a beta-positive emitter (av) (0.396 MeV) with a half-life extending to 3.3 days. ⁸⁹Zr has potential applications in positron emission tomography (PET) imaging and when included in a protein conjugate (such as those produced by the methods of the invention) is of particular interest in immunological PET (immuno-PET) imaging due to its extended 3.3 d half-life which matches the circulation half-life of an antibody. In immuno-PET imaging, tumours are imaged based upon expression of tumour-associated antigens on tumour cells through the use of a radionuclide complex conjugated to an appropriate antibody.

In addition to zirconium, DFO and its analogues are also chelators of iron. Traditional processes for handling DFO through synthetic transformations, conveniently involve iron chelated-DFO. The iron chelated form of DFO serves as a protected form, binding the coordination sites of DFO and impeding their participation in some other synthetic transformations.

The iron atom chelated to the DFO chelating moiety of the conjugate may be any suitable form. For example, the iron may be iron (II) or iron (III) or a combination thereof. In some embodiments, the iron is iron (II). In other embodiments, the iron is iron (III).

The exposure of the free chelating ligand to the iron complexed conjugate is advantageously carried out at substantially neutral pH. The inventors have shown (see Examples) that maintaining a substantially neutral pH during this step of the process avoids aggregate formation, and hence significantly improves yields. In some embodiments exposing the free chelating ligand to the iron complexed conjugate occurs at a pH from about 7 to about 8. In some embodiments, the substantially neutral pH is physiological pH, typically about 7.4. In some embodiments, the pH is about 7.2. As the conjugate comprises a peptide which may already have adopted a tertiary structure, maintaining substantially neutral pH (and/or physiological pH) is believed to assist avoid aggregate formation.

Exposure of the iron complexed conjugate to the free chelating ligand may substantially remove the iron bound to the conjugate. The removal of iron is in effect a transfer of iron from the DFO-chelating moiety to the free chelating ligand and as such may be referred to herein as a transchelation step or process. In some embodiments, following transchelation, the conjugate may comprise not more than about 1 ppm, 0.75 ppm, 0.5 ppm, 0.4 ppm, 0.3 ppm, 0.25 ppm or 0.2 ppm complexed iron. The conjugate substantially free of complexed iron may comprise from no detectable concentration of complexed iron to any of these concentrations, for example, from no detectable amount of complexed iron to about 1 ppm of complexed iron or from no detectable amount of complexed iron to 0.3 ppm complexed iron. The concentration of complexed iron following transchelation may be determined by inductively coupled plasma-mass spectrometry (ICP-MS).

### Protein

The protein of the conjugates produced by the methods of the invention may be any protein capable of conjugation with the desferrioxamine chelating ligand and that has the potential to form undesired aggregates.

The aggregates may be insoluble aggregates that form particulates and may precipitate from the solution in which they are formed, or the aggregates may be soluble aggregates. As used herein, the term “aggregate” will be understood to include high molecular weight (HMW) aggregates of the protein, typically multimers larger than a dimer. The aggregates may be insoluble aggregates that form particulates and may precipitate from the solution in which they are formed, or the aggregates may be soluble aggregates.

One class of DFO-protein conjugates of particular interest are those where the protein moiety is able to localise the conjugate within a subject after administration to assist with imaging, eg by PET, SPECT or other suitable imaging technique. Accordingly, in some embodiments, the protein may be a protein targeting agent.

As used herein, a “protein targeting agent” refers to any protein capable of:
1. stable conjugation with the desferrioxamine chelating group (both in free form and when chelating a nuclide, such as a radionuclide)
2. forming deleterious aggregates during handling, and
3. localising within a subject following administration (eg the protein targeting agent may localise in one or more organs, organelles, cell-types or receptor-types).

The protein targeting agent may be a polypeptide, a protein (eg an antibody and its derivatives such as nanobodies, diabodies, antibody fragments) that is able to bind to a certain biological target or to express a certain metabolic activity.

Non-limiting examples of suitable targeting agents include molecules that target VEGF receptors, analogs of bombesin or GRP receptor targeting molecules, molecules targeting somatostatin receptors, RGD peptides or molecules targeting avp3 and avP5, annexin V or molecules targeting the apoptotic process, molecules targeting estrogen receptors, biomolecules targeting the plaque, molecules targeting PSMA, molecules targeting a carbonic anhydrase (such as carbonic anhydrase IX; CAIX).

In some embodiments, the protein is an antibody or a derivative thereof, including as nanobodies, diabodies, antibody fragments and the like. In some embodiments, the protein is an antibody or antigen binding fragment thereof.

In any embodiment, the protein is an antibody or antigen binding fragment thereof, for binding to carbonic anhydrase IX (CAIX). A preferred antibody is cG250, preferably girentuximab (INN), also referred to herein as GmAb. Another especially preferred embodiment is the monoclonal antibody G250 produced by the hybridoma cell line DSM ACC 2526. The antibody cG250 is an IgG1 kappa light chain chimeric version of an originally murine monoclonal antibody mG250. The antibody of antigen binding fragment thereof may also be a humanised form of girentuximab. In particularly preferred embodiments, the antibody for binding to CAIX is one that is described in WO 2021/000017, the contents of which are hereby incorporated by reference.

In some embodiments, the protein is a polypeptide. The polypeptide may comprise a minimum sequence of at least about 20, 25 or 30 amino acid residues. The polypeptide may comprise up to about 35, 40, 45 or 50 amino acid residues. The polypeptide may comprise any amino acid sequence length from any of these minimum values to any maximum value, including for example about 20 to about 50 amino acid residues. Aggregation of peptides has been reviewed in Zapadka K L, Becher F J, Gomes dos Santos A L, Jackson S E. 2017 Factors affecting the physical stability (aggregation) of peptide therapeutics. Interface Focus 7: 20170030. http://dx.doi.org/10.1098/rsfs.2017.0030, which is entirely incorporated herein by reference.

In some embodiments, the protein is a native protein and is isolated from its source. In some embodiments, the protein is synthetic or semi-synthetic. The protein may be prepared by any means known in the art, including direct amino acid synthesis, recombinant technologies, and ligation of fragments to form the desired protein.

### Linker

The conjugate comprises the protein linked with the DFO-chelating agent. In some embodiments, the protein and DFO-chelating agent are linked directly through a covalent bond. In some embodiments, the protein and DFO-chelating agent are linked through a linking group.

In some embodiments, the linking group is a bifunctional linker. The bifunctional linker may be any diradical species capable of covalently linking the DFO-chelating agent and the protein together.

Suitable bifunctional linkers include bromoacetyl, thiols, succinimide ester (eg succinyl), tetrafluorophenyl (TFP) ester, a maleimide, amino acids (including natural and non-natural amino acids), a nicotinamide, a nicotinamide derivative, or using any amine or thiol-modifying chemistry known in the art.

In some embodiments, the bifunctional linker is succinyl.

In some embodiments, the bifunctional linker comprises a chain of atoms defining a longest linear path of 2-10 atoms between the DFO chelating moiety and the protein.

In some embodiments, the bifunctional linker may be a C₁₋₁₀alkyl or haloC₁₋₁₀alkyl optionally interrupted by one or more groups selected from: —O—, —NR—, —S—, —C(O)—, —C(O)O—, —C(O)NR—, —OC(O)—, —NRC(O)—, —OC(O)O—, —NRC(O)O—, —OC(O)NR—, —NRC(O)NR—, wherein R is selected from H and C₁₋₄alkyl.

In some embodiments, the bifunctional linker may comprise a chelating moiety. In these embodiments, the chelating moiety of the linker may bind a different radio nuclide(s) to the DFO-chelating ligand. In some embodiments, the chelating moiety may be a 6-hydrazinylnicotinamide moiety. The 6-hydrazinylnicotinamide moiety typically binds technetium-99 (^{99m}Tc).

### Free Chelating Ligand

The processes described herein involve exposing an iron bound form of a DFO-chelating ligand-protein conjugate with free chelating ligand. The DFO-chelating moiety is conjugated with the protein in its iron bound form to effectively protect the DFO-chelating moiety from undesired chemical reaction during the conjugation step.

Preferably the free chelating ligand has a higher affinity for iron than EDTA. Accordingly, typically the free chelating ligand is other than EDTA.

Also preferably the free chelating ligand has an equivalent or better affinity for iron than the DFO-chelating moiety of the conjugate.

EDTA is a widely used iron chelator. Its use has been described to bind iron in chemical transformations and to remove iron from solution, including in biological systems for example as chelation therapy. However, disruption of the chelation of iron by the DFO chelating moiety of the protein conjugates described herein required elevated temperatures and low pH (Examples 1 and 2). These reaction conditions resulted in the formation of irreversible aggregation of the protein conjugates leading to a significant loss of yield. Surprisingly, the inventors found that exposing the iron-bound conjugate intermediates to chelating ligands other than EDTA avoided the need for elevated temperatures and allowed the reaction to proceed at ambient temperature. Further study revealed that even at elevated temperatures, conducting the reaction at substantially neutral pH avoided the formation of aggregates (Example 2).

The free chelating ligand is a chelator of iron. The free chelating ligand may be bi-, tri-, tetra-, penta-, hexa-, septa- or octa-dentate.

The free chelating ligand may be selected from DFO, a DFO analogue and N,N-bis(2-Hydroxybenzyl)ethylenediamine-N,N-diacetic acid (HBED), or a combination thereof.

In some embodiments, the free chelating ligand is DFO or an analogue thereof.

In some embodiments, the free chelating ligand is HBED.

In some embodiments, the free chelating ligand is selected from the group consisting of: TMT (6,6″-bis[N,N″,N′″-tetra(carboxymethyl)aminomethyl)-4′-(3-amino-4-methoxyphenyl)-2,2′:6′,2″-terpyridine), DOTA (1,4,7,10-tetraazacyclododecane-N,N′,N″,N′″-tetraacetic acid, also known as tetraxetan), TCMC (the tetra-primary amide of DOTA), DO3A (1,4,7,10-Tetraazacyclododecane-1,4,7-tris(acetic acid)-10-(2-thioethyl)acetamide), CB-DO2A (4,10-bis(carboxymethyl)-1,4,7,10-tetraazabicyclo[5.5.2]tetradecan), NOTA (1,4,7-triazacyclononane-triacetic acid) Diamsar (3,6,10,13,16,19-hexaazabicyclo[6.6.6]eicosane-1,8-diamine), DTPA (Pentetic acid or diethylenetriaminepentaacetic acid), CHX-A″-DTPA ([(R)-2-Amino-3-(4-isothiocyanatophenyl)propyl]-trans-(S,S)-cyclohexane-1,2-diamine-pentaacetic acid), TETA (1,4,8,11-tetraazacyclotetradecane-1,4,8,11-tetraacetic acid), Te2A (4,11-bis(carboxymethyl)-1,4,8,11-tetraazabicyclo[6.6.2]hexadecane), HBED (N,N-bis(2-Hvdroxybenzyl)ethylenediamine-N,N-diacetic acid), DFO (Desferrioxamine), and analogues or derivatives thereof such as DFO* and DFOsq (DFO-squaramide), HYNIC (6-hydrazinonicotinamide), and HOPO (3,4,3-(LI-1,2-HOPO), or other ligand as described herein, or a derivative thereof. Suitable derivatives include modification to non-coordinating portions of the molecule and may include functional group interconversion, such as the presence of an amide in place of a carboxyl group.

In some embodiments, the free chelating ligand matches the DFO-chelating moiety included in the conjugate, for example, the DFO-chelating moiety may be DFO* and in these embodiments, the free chelating ligand may then also comprise DFO*.

The free chelating ligand may be used in a molar excess relative to the iron-chelated conjugate. Increasing the number of equivalents of free chelating ligand may assist disruption of the iron chelation by taking advantage of equilibration of iron chelation between the conjugate bound chelator and the free chelator. However, it is desirable to avoid unnecessary excess of reagent from a cost perspective and due to the potential complication of purification of the reaction product.

In some embodiments, the minimum number of molar equivalents of the free chelating ligand to the iron-chelated conjugate may be at least about 50, 55, 60, 65, 70, 75, 80, 90,100,110,120,130,140,150, 200, 250, 300, 250, 400, 450, 500, 550, 600, 650, 700, 750, 800, 850, 900, 950, 1000, 1100, 1200, 1300, 1400 or 1500 molar equivalents. The maximum number of molar equivalents of the free chelating ligand to the iron chelated conjugate may be not more than about 5000, 4750, 4500, 4250, 4000, 3750, 3500, 3250, 3000, 2750, 2500, 2250, 2000, 1750, 1500, 1250, 1000, 750, 500, 450, 400, 350, 300, 275, 250, 225, 200, 175 or 150 molar equivalents. The number of equivalents of the free chelating ligand to the iron chelated conjugate may be from any of these minimum amounts to any of these maximum amounts provided the minimum selected is lower than the maximum. For example, in some embodiments, the number of molar equivalents of the free chelating ligand is from about 50 to about 5000, about 100 to about 200, or about 1200 to about 2000 molar equivalents relative to the iron chelated conjugate.

The number of molar equivalents may alternatively be expressed as a molar ratio. A molar excess of 50 for the free chelating ligand compared to the iron bound conjugate may be expressed as a molar ratio of 1:50 iron-bound conjugate: free chelating ligand. Accordingly, in some embodiments, the minimum ratio of the iron complexed conjugate to the free chelating ligand may be at least about 1:50, 1:55, 1:60, 1:65, 1:70, 1:75, 1:80, 1:90, 1:100, 1:110, 1:120, 1:130, 1:140, 1:150, 1:200, 1:250, 1:300, 1:250, 1:400, 1:450, 1:500, 1:550, 1:600, 1:650, 1:700, 1:750, 1:800, 1:850, 1:900, 1:950, 1:1000, 1:1100, 1:1200, 1:1300, 1:1400 or 1:1500. The maximum ratio of the iron complexed conjugate to the free chelating ligand may be not more than about 1:5000, 1:4750, 1:4500, 1:4250, 1:4000, 1:3750, 1:3500, 1:3250, 1:3000, 1:2750, 1:2500, 1:2250, 1:2000, 1:1750, 1:1500, 1:1250, 1:1000, 1:750, 1:500, 1:450, 1:400, 1:350, 1:300, 1:275, 1:250, 1:225, 1:200, 1:175 or 1:150 molar equivalents. The ratio of the iron complexed conjugate to the free chelating ligand may be from any of these minimum ratios to any maximum ratio provided the minimum ratio is less than the maximum ratio, for example from about 1:50 to about 1:5000, about 1:100 to about 1:200 or about 1:1200 to about 1:2000.

The free chelating ligand may be combined with the iron complexed conjugate in any suitable form. In some embodiments, the free chelating ligand may conveniently be provided as a liquid composition. This liquid composition may comprise a liquid carrier, such as a solvent. Typically the liquid composition is a solution of the free chelating ligand in water. The minimum concentration of free chelating ligand in the liquid composition may be at least about 1 mM, 5 mM, 10 mM, 20 mM, 30 mM, 40 mM, 50 mM, 60 mM or 65 mM. The maximum concentration of free chelating ligand in the liquid composition may be not more than about 200 mM, 175 mM, 150 mM, 125 mM, 100 mM, 90 mM, 80 mM, 75 mM or 70 mM. The concentration of free chelating ligand in the liquid composition may be from any of these minimum concentrations to any of these maximum concentration, for example from about 1 mM to about 200 mM or from about 65 mM to about 70 mM.

In some embodiments, the exposure of the free chelating ligand to the iron-complexed conjugate may be at ambient temperature. Typically ambient temperatures may be from about 15° C. to about 30° C., about 20° C. to about 25° C. or at about 20° C.+/−about 5° C. Ambient temperatures are preferred as typically the preceding conjugating step is carried out at ambient temperature and minimising the temperature changes decreases the overall process time and may also assist avoid aggregate formation on larger scales (eg from multi-gram scale, 100s of gram scale or kilogram scale).

In some embodiments, the process comprises exposing an iron complexed conjugate to a free chelating ligand in the presence of unconjugated iron complexed DFO chelating moiety that is not conjugated to a protein, and may comprise a functional group activating the DFO chelating moiety for conjugation with a protein (such as a pentafluorophenyl ester, a mixed anhydride or an acid halide) or a functional group capable of being activated for conjugation (such as a carboxylic acid, a C₁₋₆alkylOC(O)—, or an amide).

### Further Steps

In some embodiments, the process further comprises a step of purifying the conjugate following the exposing step. In some embodiments, this purifying step is the only purification step included in the process. The purification step may comprise separating the conjugate from free chelating ligand, iron-complexed free chelating ligand and optionally unconjugated DFO-chelating moiety. The conjugate may be retained in a solution further comprising a solvent following purification. The solvent may be selected from ACN, DMSO, water or a combination thereof. The purification step typically comprises purifying the conjugate by column chromatography, for example a PD-10 column or a tangential flow filtration (eg ultrafiltration/diafiltration (UF/DF)). In some embodiments, the processes may comprise purification comprising one or more filtration steps (eg PD-10 column) and UF/DF. In these embodiments, the purification may comprise a first filtration step (eg filtration with a PD-10 column), a UF/DF process and a second filtration step (eg filtration with a PD-10 column).

The processes may optionally comprise one or more further filtration steps to remove any particulate matter and/or reduce bioburden.

Any step of the processes describes herein may comprise mixing, for example by magnetic stir-bar or overhead mechanical stirrer.

The processes may comprise a step of adjusting the concentration of the free-DFO-protein conjugate. The target final concentration will depend on the nature of the protein, and the skilled addressee will appreciate what concentration is desirable for handling the free-DFO-protein conjugate. In some embodiments where the protein is an antibody, the target concentration may be from about 1.8 mg/ml to about 2.2 mg/ml.

### Specific Embodiments

In another aspect, also provided is a process of preparing a conjugate of a desferrioxamine chelating ligand linked with a protein, the process comprising:
conjugating an iron chelated desferrioxamine chelating ligand with a protein to provide an iron complexed conjugate of the desferrioxamine chelating ligand complexed to iron linked with the protein; and
exposing the iron complexed conjugate comprising the desferrioxamine chelating ligand complexed to iron linked with the protein, to a free chelating ligand selected from desferrioxamine, a desferrioxamine analogue and HBED, or a combination thereof, at substantially neutral pH to thereby remove iron from the desferrioxamine chelating ligand of the iron chelated conjugate and provide the conjugate of the desferrioxamine chelating ligand linked with the protein that is substantially free of chelated iron.
   In some embodiments, the pH of the conjugating step and the exposing step is substantially the same.
   In some embodiments, the pH of the conjugating step and the exposing step is from about 7 to about 8, preferably about 7.2 or about 7.4.
   In some embodiments, the protein is girentuximab.
   In some embodiments, the desferrioxamine chelating ligand and the protein are linked through a succinyl linking group.
   In some embodiments, the desferrioxamine chelating ligand is desferrioxamine.
   In some embodiments, the iron chelated desferrioxamine chelating ligand is provided in a composition further comprising a polar organic solvent, preferably selected from ACN, DMSO or a combination thereof.
   In some embodiments, iron chelated desferrioxamine chelating ligand is provided in a molar excess relative to the protein.
   In some embodiments, the free chelating ligand is provided in a molar excess relative to the iron complexed conjugate. The molar excess may be any of those described herein.
   In some embodiments, the processes do not comprise a purification step(s) between the conjugation and the transchelation (or exposing) steps.
   In some embodiments, the conjugating and exposing steps are carried out at ambient temperatures (for example from about 18-25° C.).

### Conjugates Obtainable by Processes Described Herein

Another aspect relates to a conjugate comprising a DFO-chelating moiety conjugated to a protein obtainable or produced by the processes described herein.

Another aspect provides a composition comprising the conjugate obtainable or produced by the processes described herein.

The conjugate may be characterised by a minimum conjugate to protein ratio of at least about 0.2, 0.25, 0.3, 0.4, 0.5, 0.6, 0.7, 0.8 or 0.9. The conjugate may be characterised by a maximum conjugate to protein ratio of not more than about 4, 3.5, 3, 2.5, 2, 1.5, 1.4, 1.3, 1.2, 1.1, 1 or 0.9. The conjugate to protein ratio of the conjugate may be from any of these minimum ratios to any of these maximum ratios provided the minimum is less than the maximum. For example, the conjugate to protein ratio may be from about 0.2 to about 4 or from about 0.9 to about 1. Typically, the conjugate to protein ratio are most influenced by the conditions of the conjugation step in processes comprising this step.

The composition comprising the conjugate substantially free of iron may comprise not more than about 1%, 0.9%, 0.8%, 0.7%, 0.6%, 0.5%, 0.4%, 0.3%, 0.2%, 0.1%, 0.05%, 0.01%, 0.005%, 0.001%, 0.0005%, 0.0001% or lower concentration of high molecular weight (HMW) protein aggregates relative to the monomeric conjugate. The composition comprising the conjugate may comprise HMW aggregates from any of these percentages to any other percentage, for example, from about 0.01% to about 1%, preferably 0.0001% to about 0.1%. The concentration of HMW species relative to the monomeric conjugate may be determined by SEC-HPLC and comparison of the area under the peak for the monomeric protein compared with the area under the peak attributable to the HMW species. One example of a suitable SEC-HPLC protocol is described for DFO-succinyl-girentuximab conjugates in the Examples.

In some embodiments, the composition may further comprise dimeric conjugate. Typically the dimer is present in a concentration of not more than about 1%, 0.9%, 0.8% or 0.7%. The dimer may be present between any of these concentrations, for example from about 0.7% to about 1%. The concentration of dimer may be determined in a similar manner to the concentration of HMW species, for example by SEC-HPLC and comparison of the area under the peak for the respective relevant peaks.

The composition comprising the conjugate may comprise a solvent. The solvent may be any solvent described herein. In some embodiments, the solvent may be selected from ACN, DMSO, water and combinations thereof. For some proteins, aggregation has been known to occur under higher concentrations. A composition comprising a conjugate intended for radiolabelling should possess a suitable concentration of conjugate for the radiolabelling step, and preferably for subsequent administration to a subject. Accordingly, in some embodiments, the maximum concentration of conjugate in a composition comprising the conjugate substantially free of iron and solvent may be up to about 10, 7.5, 5, 4, 3, 2, 2.5, 2.4, 2.3, 2.2 mg/ml. The minimum concentration of conjugate in a composition comprising the conjugate substantially free of iron and a solvent may be at least about 0.5, 1, 1.5 or 1.8 mg/ml. For example, the concentration of conjugate in such compositions may be from about 1 to about 10 mg/mL, about 1.5 mg/ml to about 5 mg/ml, about 1.5 mg/ml to about 5 mg/ml, or about 1.8 to about 2.2 mg/mL.

### EXAMPLES

### Definitions

% CV: Coefficient of variation: Standard deviation divided by the mean expressed as percentage. Syn.: % RSD (relative standard deviation)
A280: Absorbance at 280 nm
A428: Absorbance at 428 nm
ACN: Acetonitrile
BDS: Bulk Drug Substance
BSA: Bovine serum albumin
CAR: Chelate-to-antibody ratio
CE: Capillary electrophoresis. Also iCE: Imaged capillary electrophoresis, and CESDS: Capillary electrophoresis (with) sodium dodecyl sulfate
cIEF: Capillary isoelectric focusing. Also icIEF: Imaged capillary isoelectric focusing
DCU: Digital control unit
DFO: desferrioxamine (syn.: deferoxamine, or Desferal)
DMSO: Dimethyl Sulfoxide
PBS: Phosphate Buffered Saline
DS: Drug substance
ELISA: Enzyme-linked immunosorbent assay
ESI: Electrospray ionization
GmAb: Chimeric girentuximab, syn. WX-G250, unmodified antibody
GmAb-DFO: Chimeric girentuximab-N-succinyldesferal conjugate, syn.: GmAb-N-sucDf
GmAb-DF-Fe: The intermediate conjugate before removing iron (III) to generate GmAb-DFO as drug product
HBED: N,N′-Di(2-hydroxybenzyl)ethylenediamine-N,N′-diacetic acid monohydrochloride
IgG: Immunoglobulin G
kDa: Kilodalton
LC: Liquid chromatography
MS: Mass spectrometry
MW: Molecular weight
PD: Process development
PDA: Photodiode array. Syn.: DAD—diode array detector
QTOF: Quadrupole time-of-flight
QS: Quantum satis
RM: Reference material
RCP: Radio chemical purity
RPM: Revolution per min
SDS: Sodium dodecyl sulfate
SEC: Size exclusion chromatography; see also SE-HPLC: Size exclusion high performance liquid chromatography
SOP: Standard operating procedure
SR: Substitution ratio. Syn.: chelator-to-antibody ratio (CAR)
Syn.: Synonym
TFP-N-sucDf-Fe:N-succinyldesferal: Fe(III) tetrafluorophenyl ester, (syn.: DFOTFP); a bifunctional chelator
USP: United States Pharmacopeia
WFI: Water for irrigation
Zr-89: Syn. ⁸⁹Zr, a positron-emitting radionuclide

### Example 1—Preparation of Iron Chelated Conjugate of DFO and Girentuximab (GmAb-N-sucDf-Fe)

This example describes the preparation of an iron chelated conjugate of DFO linked to girentuximab through a succinyl linking group, and an optimisation study of the conjugation reaction of girentuximab (GmAb) and tetrafluorophenyl-N-succinyldesferrioxamine iron chelate to provide a DFO-girentuximab conjugate. The procedures are a mix of standard processes and use of an automated reaction system. Any step performed in the automated reactor could alternatively be carried out in conventional equipment, and similarly the entire process could be adapted to be carried out in an automated system by adjusting the scale of each step.

### Starting Materials

Girentuximab (GmAb) antibody, 4.91 mg/mL, stored at 2-8° C.

Bifunctional chelator: tetrafluorophenyl-N-succinyldesferrioxamine iron chelate (TFP-N-sucDf-Fe), stored at −20±5° C.

### Procedure

### Preparation of GmAb Solutions

A 4.91 mg/ml solution of GmAb in 10 mM aq. sodium phosphate, 150 mM aq. NaCl, pH 7.2 provides the GmAb starting pool. All solutions once prepared were filtered through a 0.2 μm filtration device prior to use. The GmAb starting pool has a pH of 7.2, and a portion of the GmAb starting pool was divided and titrated to pH 8.4 or 9.6 to provide a GmAb pH adjusted pool (sometimes referred to herein as pH adjusted GmAb pool). The concentration of the GmAb pH adjusted pool can be calculated based on the combined volume or the GmAb starting pool and the volume of base added, or the concentration can be experimentally determined by UV-vis spectrophotometry (see protocol below).

### Preparation of TFP-N-sucDf-Fe Solutions

Solutions of TFP-N-sucDf-Fe were prepared in each of the selected solvents (ACN or DMSO) at 3 different concentration levels so that the number of equivalents could be varied without impacting the overall volume of solvent in each vessel:
(i) 2.5 mg/ml to provide 3 molar equivalents of TFP-N-sucDf-Fe relative to GmAb in the following conjugation step
(ii) 6.25 mg/ml to provide 7.5 equivalents
(iii) 10.0 mg/ml to provide 12 equivalents.

### Conjugation of TFP-N-sucDf-Fe and GmAb

Each of the TFP-N-sucDf-Fe solutions was added to a separate vessel containing GmAb starting pool or GmAb pH adjusted pool. The mixing speed of each reaction vessel was maintained for the duration of the addition, then reduced for the incubation period. The solution at the end of the incubation time provides a Conjugated Product Pool containing GmAb-N-subDf-Fe in solution (conjugated pool). The conjugated product pool was titrated (if required) to a target pH of 7.0 with to provide a pH adjusted conjugated product pool (sometimes referred to herein as conjugated pH adjusted pool). The colour of the reaction mixtures at this stage of the process ranged from pale orange to dark orange. Reaction mixtures became darker in orange colour as the mole ratio of TFP-N-sucDf-Fe:GmAb increased from 3:1 to 12:1. The appearance of all reaction mixtures were clear and free of particulates.

Removal of Fe from GmAb-N-sucDf-Fe

The conjugated pH adjusted pool was heated to 35° C. (heated conjugated pool, or temperature adjusted conjugated product pool). The heated solution was titrated to a pH of 4.4-4.5 to provide a Low pH Conjugated Product Pool. Chelated iron was removed by addition of 25 mg/ml disodium EDTA aqueous solution pre-warmed to 35° C. at a ratio of 15 mL EDTA solution per gram of GmAb and incubation for 110 minutes while stirring to provide a transchelated product pool. Progress of this reaction was monitored by absorbance measurement at 428 nm. Typically, the transchelation solutions remained clear for up to 40 min and then the solutions began to appear slightly opalescent. There were no visible particulates observed for any samples over the course of the 110 min. Following incubation, the transchelated product pool was titrated to a pH of 6.8-7.2 to provide a Neutralised Transchelated Product Pool. The target product was purified by PD-10 column chromatography to provide a PD-10 product pool. For all samples, after equilibration of the column with 0.9% sodium chloride, 1.9 mL of sample was added to the PD-10 column and the sample was stacked with addition of 0.6 mL 0.9% sodium chloride to obtain loading volume of 2.5 mL, and then additional mobile phase was run through the PD-10 column and the exiting solution was collected in clean vessels.

Each PD-10 purified product pool was filtered through a 0.22 μm regenerated cellulose syringe filter. The filtrate was collected into clean, sterile containers to obtain the Bulk Drug Substance (BDS).

The yields assessed by A280 were all greater than 87%.

The colour of the reaction mixtures at this stage of the process ranged from colorless to faint yellow. Reaction mixtures became more yellow in color as the mole ratio of linker:GmAb increased from 3:1 to 12:1. The appearance of all reaction mixture were clear to slightly opalescent and free of particulates.

### Determination of Conjugate-to-Antibody Ratio (CAR) by LC-MS

The CAR was determined by LC-MS for selected BDS samples. Testing was performed using a minimum of 200 μL sample volume at approximately 2 mg/mL. The target CAR value for the GmAb-N-sucDf conjugate was 0.9.

Determined CAR values ranged from 0.59 to 4.10. CAR values increased when carrying out the conjugation under alkaline conditions. Of the CAR values obtained, an increase in CAR was observed when the pH of the conjugation step was increased from pH 7.2 to 8.4 or 9.6 (mole ratio of TFP-N-sucDf-Fe:GmAb being held constant). The lowest CAR value of 0.59 was obtained for conjugation conducted under neutral pH conditions (TFP-N-sucDf-Fe:GmAb mole ratio of 3:1), while CAR values of 1.26 and 1.16 were observed at pH levels of 8.4 and 9.6, respectively at the same TFP-N-sucDf-Fe:GmAb mole ratio.

A CAR value as high as 4.10 was obtained when the conjugation was performed at a TFP-N-sucDf-Fe:GmAb mole ratio of 12:1. At a mole ratio of 7.5:1, CAR values ranged from 1.88 to 2.80 depending on pH.

Based on the available CAR data, the preferred TFP-N-sucDf-Fe:GmAb mole ratio is 3:1 as CAR values ranged from 0.59 to 1.26.

### Determination of the Concentration of Girentuximab (GmAb) by Nanodrop Spectrophotometer

System suitability measurements were performed on the Nanodrop using a bovine Serum Albumin (BSA) Standard. The system suitability measurements passed all acceptance criteria of BSA concentration within the range of 0.95-1.05 mg/mL at the beginning and end of each run confirming that the NanoDrop instrument was performing suitably each day of the study.

The Nanodrop was blanked using PBS Buffer, pH 7.1.

The O.D. (A280) of the GmAb solution was measured by Nanodrop spectrophotometry. The measured A280 value was 6.629.

The concentration of the GmAb starting material was calculated using the extinction coefficient of 1.35 (mg/mL)⁻¹ cm⁻¹ and the following equation (1):

$$GmAb Concentration , \frac{mg}{ml} = \frac{A 280 Value}{( 1.35 \frac{{mg}^{- 1}}{mL} {cm}^{- 1} ) * ( 1 cm )} \qquad ( 1 )$$

The GmAb concentration in the GmAb starting pool was determined to be 4.91 mg/ml.

The total GmAb protein available was calculated by multiplying the concentration of calculated GmAb (4.91 mg/mL) by the volume of each reaction (2.3 mL) and by the total number of reactions to be performed each day. Three (3) reactions were performed each day, so the amount of GmAb was calculated as 33.9 mg of GmAb for all days.

The total volume of GmAb pool was calculated by multiplying the volume of each reaction (2.3 mL) by the total number of reactions to be performed each day.

A similar protocol was applied to assess the concentration of GmAb, and GmAb-containing conjugates in samples obtained throughout the process using absorbance detection at 280 nm (A280).

### Conclusion

Conjugation conditions for the generation of high monomeric purity GmAb-DFO conjugates with CAR values close to 0.9 were investigated. The parameters investigated included pH at three levels (7.2, 8.4, and 9.6), mole ratio of TFP-N-sucDf-Fe:GmAb at three levels (3:1, 7.5:1, and 12:1), and solvent (acetonitrile and DMSO).

The degree of conjugation tended to increase under more alkaline conditions compared to neutral pH conditions. At a TFP-N-sucDf-Fe:GmAb mole ratio of 3:1, CAR values were lowest at neutral pH (CAR 0.78 and 0.59 using linker solution in acetonitrile and DMSO, respectively). Under more alkaline pH levels at the same linker:GmAb mole ratio (3:1), CAR values were all above 1.0 and reached up to a CAR value of 1.26 (pH 8.4 using TFP-N-sucDf-Fe solution in DMSO).

The degree of conjugation also increased under conditions in which larger mole ratios of TFP-N-sucDf-Fe:GmAb were employed. At neutral pH and a mole ratio of 12:1, CAR values reached 2.97 and 2.79 using linker solution in acetonitrile and DMSO, respectively. Under more alkaline conditions, CAR values reached as high as 4.10 (mole ratio 12:1 and pH 8.4).

In some cases, the formation of HMW aggregates (70% relative retention time compared to the monomer peak “RRT”) tended to increase, albeit modestly, when the mole ratio of TFP-N-sucDf-Fe:GmAb was increased from 3:1 to 12:1. The overall monomeric purity of the BDS in all cases were within acceptable limits. However, it has been shown the level of HMW aggregate formation at larger scales can be problematic as observed in previous manufacturing campaigns. The levels of HMW aggregate generated at small scale do not tend to correlate to the levels of HMW aggregate generated under the same conditions at larger scale. Therefore the relative amount of HMW aggregate is expected to increase at larger scales, translating to significant concentrations of HWM aggregates and corresponding loss of yield.

Given the results of this study, a mole ratio of 3:1 TFP-N-sucDf-Fe:GmAb is ideal to obtain CAR values close to 0.9. At pH 9.6, the CAR value obtained was 1.10. However, even under neutral pH conditions, a CAR value of 0.78 can be achieved. While good CAR values can be obtained under alkaline conditions, neutral pH conditions provide acceptable CAR while providing a more streamlined process (fewer titration steps required) while maintaining acceptable conditions to avoid significant aggregation.

The results from this example suggested that the three (3) best conjugation conditions may employ the following conjugations conditions: (1) pH 7.2, Mole Ratio 3:1, TFP-N-sucDf-Fe in DMSO, (2) pH 7.2, Mole Ratio 3:1, TFP-N-sucDf-Fe in ACN, and (3) pH 8.4, Mole Ratio 3:1, TFP-N-sucDf-Fe in ACN.

These three (3) conditions as well as BDS (prepared at pH 9.6, Mole Ratio 3:1, TFP-N-sucDf-Fe in ACN) were used as starting material for Example 2.

These conjugation products were used to evaluate additional conjugation reaction parameters including the TFP-N-sucDf-Fe addition rate, the TFP-N-sucDf-Fe addition concentration, and the TFP-N-sucDf-Fe manufacturer in Example 2. The responses measured include the % High Molecular Weight (HMW) species by SEC-HPLC, the chelate-to-antibody ratio (CAR) by LC/MS, and the yield of the reaction measured by A280.

### Example 2—Improvement of Removal of Fe from GmAb-NsucDf-Fe

BDS prepared as described in Example 1 were used as starting materials for the studies described in this Example. The reaction mixture was split into three equal volumes.

One vessel utilized the conditions similar to those described in Example 1 (EDTA, pH 4.4, and 35° C.). Under these conditions, the transchelation process was monitored by A428 according to the protocol outlined in Example 1.

A second vessel was evaluated by performing the transchelation using DFO mesylate at ambient temperature and neutral pH (pH 7.0). Under these conditions, the transchelation was monitored by A280 and A428, although no colour change was expected.

A third vessel was evaluated by performing the transchelation using HBED (N,N′-Di(2-hydroxybenzyl)ethylenediamine-N,N′-diacetic acid monohydrochloride) at ambient temperature and neutral pH. The transchelation was monitored by A428, however HBED does absorb at 428 nm.

The conjugates were purified using PD-10 columns and characterized by SEC-HPLC for monomeric purity, A280 for process yield, and radiochemical purity to determine the efficiency of the transchelation process.

The screening reactions tested in this example are summarized in Table 1.

TABLE 1
Experimental Design for Phase IA and Phase II

| | | | | TFP-N-sucDf-Fe | | | | | |
|---|---|---|---|---|---|---|---|---|---|
| | Conjugation | | | Addition | | | Transchelation Conditions | | |
| Reaction | | Mole | | Concentration | Addition | TFP-N-sucDf-Fe | Transchelating | pH | Temperature |
| # | pH | Ratio | Solvent | (mg/mL) | Time (min) | Manufacturer | Agent | (±2° C.) | (±2° C.) |
| 1 | 9.6 | 3 | ACN | 2.50 | 2 | ERAS | EDTA | 4.4 | 35 |
| 2 | 9.6 | 3 | ACN | 2.50 | 2 | ERAS | DFO | 7.0 | 22 |
| 3 | 9.6 | 3 | ACN | 2.50 | 2 | ERAS | HBED | 7.0 | 22 |
| 4 | 7.2 | 3 | DMSO | 2.50 | 2 | ERAS | EDTA | 4.4 | 35 |
| 5 | 7.2 | 3 | DMSO | 2.50 | 2 | ERAS | DFO | 7.0 | 22 |
| 6 | 7.2 | 3 | DMSO | 2.50 | 2 | ERAS | HBED | 7.0 | 22 |
| 7 | 7.2 | 3 | ACN | 2.50 | 2 | ERAS | EDTA | 4.4 | 35 |
| 8 | 7.2 | 3 | ACN | 2.50 | 2 | ERAS | DFO | 7.0 | 22 |
| 9 | 7.2 | 3 | ACN | 2.50 | 2 | ERAS | HBED | 7.0 | 22 |
| 10 | 8.4 | 3 | ACN | 2.50 | 2 | ERAS | EDTA | 4.4 | 35 |
| 11 | 8.4 | 3 | ACN | 2.50 | 2 | ERAS | DFO | 7.0 | 22 |
| 12 | 8.4 | 3 | ACN | 2.50 | 2 | ERAS | HBED | 8.4 | 22 |
| 13 | 9.6 | 3 | DMSO | 2.50 | 2 | ABX | EDTA | 4.4 | 35 |
| 14 | 9.6 | 3 | ACN | 5.00 | 2 | ERAS | EDTA | 4.4 | 35 |
| 15 | 9.6 | 3 | ACN | 1.25 | 2 | ERAS | EDTA | 4.4 | 35 |
| 16 | 9.6 | 3 | ACN | 2.50 | 1 | ERAS | EDTA | 4.4 | 35 |
| 17 | 9.6 | 3 | ACN | 2.50 | 5 | ERAS | EDTA | 4.4 | 35 |
| 18 | 7.2 | 3 | DMSO | 2.50 | 2 | ABX | EDTA | 4.4 | 35 |
| 19 | 7.2 | 3 | ACN | 5.00 | 2 | ERAS | EDTA | 4.4 | 35 |
| 20 | 7.2 | 3 | ACN | 1.25 | 2 | ERAS | EDTA | 4.4 | 35 |
| 21 | 7.2 | 3 | ACN | 2.50 | 1 | ERAS | EDTA | 4.4 | 35 |
| 22 | 7.2 | 3 | ACN | 2.50 | 5 | ERAS | EDTA | 4.4 | 35 |
| 23 | 9.6 | 3 | ACN | 2.50 | Hold 5 min | ERAS | EDTA | 4.4 | 35 |
| | | | | | Add 2 min | | | | |
| 24 | 9.6 | 3 | ACN | 2.50 | Hold 10 min | ERAS | EDTA | 4.4 | 35 |
| | | | | | Add 2 min | | | | |
| 25 | 9.6 | 3 | ACN | 2.50 | Hold 15 min | ERAS | EDTA | 4.4 | 35 |
| | | | | | Add 2 min | | | | |
| 26 | 7.2 | 3 | DMSO | 2.50 | Hold 5 min | ERAS | EDTA | 4.4 | 35 |
| | | | | | Add 2 min | | | | |
| 27 | 7.2 | 3 | DMSO | 2.50 | Hold 10 min | ERAS | EDTA | 4.4 | 35 |
| | | | | | Add2 min | | | | |
| 28 | 7.2 | 3 | DMSO | 2.50 | Hold 15 min | ERAS | EDTA | 4.4 | 35 |
| | | | | | Add 2 min | | | | |

Chelated Iron Removal with EDTA Disodium, DFO, or HBED

Chelated iron used to stabilize the linker was removed with EDTA Disodium, DFO, or HBED (each referred to as transchelant) at a ratio of 15 mL EDTA solution per gram GmAb or 150:1 mole ratio of transchelant:GmAb.

The A280 value was measured to determine the concentration experimentally and the results are reported for each sample. The concentration by A280 is higher than the theoretical concentration and this result is likely attributed to the presence of unconjugated DFO starting material and slight turbidity of the samples.

### Addition of EDTA Disodium Solution (Pre-Warmed to 35° C.), DFO, or HBED to the Reaction Mixture

EDTA solution was confirmed to be equilibrated to 35° C. prior to initiating this step. DFO or HBED solutions were not heated and remained at ambient temperature (20-25° C.).

The mixing speed of each reaction vessel was maintained.

Using a manual pipet, each transchelant solution was added to each reaction vessel.

### Incubation of the Transchelation Reaction Mixture

Each reaction vessel was incubated at 35±2° C. for 110 min after the addition of the EDTA Disodium solution based on the results discussed in Example 1. Transchelations performed using DFO or HBED were incubated at ambient temperature (20-25° C.).

The mixing speed was maintained.

After 20, 40, 80 and 100 min of incubation, the progress of each transchelation reaction was checked by observing the color of the solution and the absorbance at 428 nm. The incubation was stopped after 110 min.

For reactions using EDTA as the transchelant, the colour changed from light orange to clear. For reactions that used DFO or HBED, no colour change was observed. Additionally, no visible particulates were observed for all reactions over the course of the 110 min incubation.

### Adjustment of pH to 6.8-7.2

To quench the transchelation reaction, the reactions were titrated to pH of 7.0±0.2 (if required). This titration was automated using the dose syringe pump and pH probe connected to the automated reactor. The reaction vessels were removed from the incubator. The mixing speed was maintained. The appearance of each reaction mixture at this stage of the process was colourless, slightly opalescent, and free of particulates.

### Purification of Neutralized Transchelated Product Pool Using PD-10 Columns

Following titration, the neutralised transchelation products were purified by column chromatography using PD-10 columns. Each PD-10 column was equilibrated with 25 mL of 0.9% Sodium Chloride. For all samples, the volume of sample was added to a PD-10 column and the sample was stacked with additional 0.9% Sodium Chloride to obtain a total loading volume of 2.5 mL. If 2.5 mL of sample was available, no 0.9% Sodium Chloride was required. The flow through volume was discarded. 3.5 mL of 0.9% Sodium Chloride was added to each PD-10 column and the solution was allowed to enter the packed bed completely. The flow through was collected in a clean collection container.

The experimental concentration using A280 value was measured for each sample to determine whether dilution was required. If dilution was required, 0.9% Sodium Chloride was used to make the concentration of each sample fall within the range 1.8-2.2 mg/mL.

For reactions that used EDTA as the transchelant, the appearance of each reaction mixture at this stage of the process was colourless, slightly opalescent to clear, and free of particulates.

For reactions that used DFO or HBED as the transchelant, the appearance of each reaction mixture at this stage of the process was light yellow, slightly opalescent to clear, and free of particulates.

### Final Filtration to Generate the Bulk Drug Substance (BDS)

Each PD-10 purified product pool was filtered through a 0.22 μm regenerated cellulose syringe filter. The filtrate was collected into clean, sterile containers to obtain the Bulk Drug Substance.

The concentration was measured using A280 to determine the final concentration of each sample.

The yields of each reaction were greater than 86%.

For reactions that used EDTA as the transchelant, the appearance of each BDS was colourless, clear, and free of particulates.

For reactions that used DFO or HBED as the transchelant, the appearance of each BDS was light yellow, clear, and free of particulates.

The resulting BDS samples were aliquoted into microcentrifuge tubes for storage at 2-8° C. or −20° C.

### Analytical Testing

### Protein Concentration by A280

The procedure described in Example 1 was followed to measure the protein concentration.

### SEC-HPLC

All samples for testing were diluted to 2 mg/mL (if required) using sample buffer. The sample buffer contained all of the sample matrix components except for the GmAb antibody.

After diluting the sample to 2 mg/mL in sample buffer, the sample was mixed at a ratio of 90% sample to 10% mobile phase.

The samples were not filtered prior to injection onto the HPLC. Instead, the samples were centrifuged at 13,000×g for 5 min. The supernatant solution was then transferred to the HPLC vial.

The temperature of the autosampler was set to 4° C.

SEC-HPLC results are provided in Table 2 below.

TABLE 2
SEC-HPLC Results 1. Reaction numbers correspond to those detailed in Table 1.

| | RXN #1 | | | | RXN #2 | | | | | | | |
|---|---|---|---|---|---|---|---|---|---|---|---|---|
| | % | | | | [symbol] | | | | RXN #3 | | | |
| PD | HMW | | [symbol] | | % HMW | | [symbol] | | % HMW | | % | |
| Step | (70% | [symbol] | % HMW | % | (70% | % | % HMW | % | (70% | | HMW | % |
| # | RRT) | % Dimer | (total) | Monomer | RRT) | Dimer | (total) | Monomer | RRT) | % Dimer | (total) | Monomer |
| 1 | 0.1 | 0 | 0 | 98.4 | 0 | 0.8 | 0 | 98.4 | 0 | 0 | 0.9 | 98.4 |
| | | 0.8 | 0.9 | | 0.1 | | 0.9 | | 0.1 | 0.8 | | |
| 2 | 0.1 | 0.8 | 0.9 | 98.4 | 0.1 | 0.8 | 0.9 | 98.4 | 0.1 | 0.8 | 0.9 | 98.4 |
| 3 | 0.1 | 0.8 | 0.9 | 98.4 | 0.1 | 0.8 | 0.9 | 98.4 | 0.1 | 0.8 | 0.9 | 98.4 |
| 4 | 0.0 | 0.8 | 0.8 | 98.5 | N/A | N/A | N/A | N/A | N/A | N/A | N/A | N/A |
| 5 | 0.1 | 0.8 | 0.9 | 98.4 | N/A | N/A | N/A | N/A | N/A | N/A | N/A | N/A |
| 6 | 0.1 | 0.8 | 0.8 | 98.4 | 0.1 | 0.7 | 0.8 | 98.5 | 0.1 | 0.7 | 0.8 | 98.5 |
| 7 | 0.1 | 0.8 | 0.9 | 98.3 | N/A | N/A | N/A | N/A | N/A | N/A | N/A | N/A |
| 8 | 0.1 | 0.8 | 0.9 | 98.4 | 0.1 | 0.8 | 0.9 | 98.4 | 0.1 | 0.7 | 0.8 | 98.5 |
| 9 | 0.2 | 0.8 | 1.1 | 98.2 | 0.1 | 0.8 | 0.9 | 98.4 | 0.1 | 0.8 | 0.8 | 98.5 |
| | RXN #4 | | | | RXN #5 | | | | RXN #6 | | | |
| | % | | | | [symbol] | | | | [symbol] | | | |
| PD | HMW | | [symbol] | | % HMW | | [symbol] | | % HMW | | % | |
| Step | (70% | | % HMW | % | (70% | % | % HMW | % | (70% | [symbol] | HMW | % |
| # | RRT) | % Dimer | (total) | Monomer | RRT) | Dimer | (total) | Monomer | RRT) | % Dimer | (total) | Monomer |
| 1 | N/A | N/A | N/A | N/A | N/A | N/A | N/A | N/A | N/A | N/A | N/A | N/A |
| 2 | 0.1 | 0.8 | 0.9 | 98.5 | 0.1 | 0.8 | 0.9 | 98.5 | 0.1 | 0.8 | 0.9 | 98.5 |
| 3 | N/A | N/A | N/A | N/A | N/A | N/A | N/A | N/A | N/A | N/A | N/A | N/A |
| 4 | 0.1 | 0.8 | 0.8 | 98.5 | N/A | N/A | N/A | N/A | N/A | N/A | N/A | N/A |
| 5 | 0.1 | 0.8 | 0.9 | 98.4 | N/A | N/A | N/A | N/A | N/A | N/A | N/A | N/A |
| 6 | 0.1 | 0.8 | 0.8 | 98.5 | 0.1 | 0.7 | 0.8 | 98.5 | 0.0 | 0.6 | 0.7 | 98.6 |
| 7 | 0.1 | 0.8 | 0.9 | 98.4 | N/A | N/A | N/A | N/A | N/A | N/A | N/A | N/A |
| 8 | 0.1 | 0.7 | 0.9 | 98.4 | 0.1 | 0.7 | 0.8 | 98.5 | 0.1 | 0.7 | 0.8 | 98.5 |
| 9 | 0.1 | 0.8 | 0.9 | 98.4 | 0.1 | 0.8 | 0.8 | 98.5 | 0.1 | 0.8 | 0.8 | 98.5 |

| | RXN #7 | | | | | | | | RXN #9 | | | |
|---|---|---|---|---|---|---|---|---|---|---|---|---|
| | % | | | | RXN #8 | | | | % | | | |
| PD | HMW | | % | | % | | % | | HMW | | % | |
| Step | (70% | % | HMW | % | HMW | % | HMW | % | (70% | % | HMW | % |
| # | RRT) | Dimer | (total) | Monomer | (70% RRT) | Dimer | (total) | Monomer | RRT) | Dimer | (total) | Monomer |
| 1 | N/A | N/A | N/A | N/A | N/A | N/A | N/A | N/A | N/A | N/A | N/A | N/A |
| 2 | 0.1 | 0.8 | 0.9 | 98.4 | 0.1 | 0.8 | 0.9 | 98.4 | 0.1 | 0.8 | 0.9 | 98.4 |
| 3 | N/A | N/A | N/A | N/A | N/A | N/A | N/A | N/A | N/A | N/A | N/A | N/A |
| 4 | 0.1 | 0.8 | 0.8 | 98.5 | N/A | N/A | N/A | N/A | N/A | N/A | N/A | N/A |
| 5 | 1.2 | 0.8 | 2.0 | 97.3 | N/A | N/A | N/A | N/A | N/A | N/A | N/A | N/A |
| 6 | 1.7 | 0.7 | 2.4 | 96.9 | 0.1 | 0.7 | 0.8 | 98.5 | 0.0 | 0.6 | 0.6 | 98.5 |
| 7 | 1.1 | 0.7 | 1.8 | 98.2 | N/A | N/A | N/A | N/A | N/A | N/A | N/A | N/A |
| 8 | 1.6 | 0.7 | 2.3 | 97.0 | 0.1 | 0.8 | 0.8 | 98.5 | 0.1 | 0.7 | 0.8 | 98.5 |
| 9 | 1.6 | 0.7 | 2.3 | 97.0 | 0.1 | 0.8 | 0.8 | 98.5 | 0.1 | 0.8 | 0.8 | 98.6 |

| | RXN #10 | | | | | | | | RXN #12 | | | |
|---|---|---|---|---|---|---|---|---|---|---|---|---|
| | % | | | | RXN #11 | | | | % | | | |
| PD | HMW | | | | % HMW | | % | | HMW | | % | |
| Step | (70% | | % HMW | % | (70% | % | HMW | % | (70% | | HMW | % |
| # | RRT) | % Dimer | (total) | Monomer | RRT) | Dimer | (total) | Monomer | RRT) | % Dimer | (total) | Monomer |
| 1 | 0.0 | 0.8 | 0.9 | 98.5 | 0.0 | 0.8 | 0.9 | 98.5 | 0.0 | 0.8 | 0.9 | 98.5 |
| 2 | 0.0 | 0.8 | 0.8 | 98.5 | 0.0 | 0.8 | 0.8 | 98.5 | 0.0 | 0.8 | 0.8 | 98.5 |
| 3 | 0.1 | 0.8 | 0.9 | 98.5 | 0.1 | 0.8 | 0.9 | 98.5 | N/A | N/A | N/A | N/A |
| 4 | 0.1 | 0.8 | 0.8 | 98.5 | N/A | N/A | N/A | N/A | N/A | N/A | N/A | N/A |
| 5 | 3.2 | 0.8 | 4.0 | 95.4 | N/A | N/A | N/A | N/A | N/A | N/A | N/A | N/A |
| 6 | 4.0 | 0.7 | 4.7 | 94.6 | 0.1 | 0.8 | 0.9 | 98.5 | 0.1 | 0.7 | 0.7 | 98.6 |
| 7 | 1.6 | 0.7 | 2.3 | 97.7 | N/A | N/A | N/A | N/A | 0.0 | 0.6 | 0.7 | 98.7 |
| 8 | 1.5 | 0.7 | 2.2 | 97.8 | 0.1 | 0.8 | 0.9 | 98.5 | 0.1 | 0.7 | 0.8 | 98.5 |
| 9 | 1.0 | 0.8 | 1.7 | 97.6 | 0.1 | 0.8 | 0.8 | 98.5 | 0.1 | 0.7 | 0.8 | 98.6 |

| | RXN #13 | | | | RXN #14 | | | | | | | |
|---|---|---|---|---|---|---|---|---|---|---|---|---|
| | % | | | | % | | | | RXN #15 | | | |
| PD | HMW | | % | | HMW | | % | | % HMW | | % | |
| Step | (70% | | HMW | % | (70% | % | HMW | % | (70% | % | HMW | % |
| # | RRT) | % Dimer | (total) | Monomer | RRT) | Dimer | (total) | Monomer | RRT) | Dimer | (total) | Monomer |
| 1 | 0.0 | 0.9 | 0.9 | 98.4 | 0.0 | 0.9 | 0.9 | 98.4 | 0.0 | 0.9 | 0.9 | 98.3 |
| 2 | 0.1 | 0.9 | 0.9 | 98.4 | 0.0 | 0.9 | 0.9 | 98.4 | 0.1 | 0.8 | 0.9 | 98.3 |
| 3 | 0.1 | 0.9 | 1.0 | 98.4 | 0.1 | 0.9 | 0.9 | 98.3 | 0.2 | 0.9 | 1.1 | 98.1 |
| 4 | 0.1 | 0.8 | 0.9 | 98.4 | 0.1 | 0.9 | N/A | 98.4 | 0.2 | 0.8 | 1.1 | 98.2 |
| 5 | 2.4 | 0.8 | 3.2 | 96.1 | 1.0 | 0.9 | N/A | 97.4 | 3.0 | 0.8 | 3.7 | 95.5 |
| 6 | 3.1 | 0.8 | 3.8 | 95.5 | 1.6 | 0.8 | 0.8 | 96.8 | 4.4 | 0.8 | 5.2 | 94.0 |
| 7 | 3.2 | 0.8 | 4.1 | 95.3 | 1.6 | 0.8 | N/A | 96.9 | 4.0 | 0.8 | 4.8 | 94.4 |

| | RXN #16 | | | | | | | | | | | |
|---|---|---|---|---|---|---|---|---|---|---|---|---|
| | % | | | | RXN #17 | | | | RXN #18 | | | |
| PD | HMW | | | | % HMW | | % | | % HMW | | % | |
| Step | (70% | | % HMW | % | (70% | % | HMW | % | (70% | % | HMW | % |
| # | RRT) | % Dimer | (total) | Monomer | RRT) | Dimer | (total) | Monomer | RRT) | Dimer | (total) | Monomer |
| 1 | 0.0 | 0.8 | 0.9 | 98.3 | 0.1 | 0.9 | 0.9 | 98.4 | N/A | N/A | N/A | N/A |
| 2 | 0.1 | 0.9 | 0.9 | 98.3 | 0.1 | 0.9 | 0.9 | 98.4 | 0.3 | 0.8 | 1.1 | 98.2 |
| 3 | 0.1 | 0.8 | 0.9 | 98.3 | 0.1 | 0.9 | 1.0 | 98.3 | N/A | N/A | N/A | N/A |
| 4 | 0.1 | 0.9 | 0.9 | 98.3 | 0.1 | 0.9 | 1.0 | 98.3 | 0.2 | 0.8 | 1.1 | 98.3 |
| 5 | 1.1 | 0.8 | 2.0 | 97.3 | 1.7 | 0.9 | 2.6 | 96.7 | 3.8 | 0.8 | 4.6 | 94.7 |
| 6 | 1.6 | 0.8 | 2.4 | 96.8 | 2.3 | 0.8 | 3.1 | 96.1 | 3.5 | 0.8 | 4.3 | 95.1 |
| 7 | 1.4 | 0.8 | 2.2 | 97.0 | 2.2 | 0.8 | 3.0 | 96.2 | 3.5 | 0.8 | 4.3 | 95.1 |
| 8 | 1.3 | 0.8 | 2.1 | 97.1 | 2.0 | 0.9 | 2.8 | 96.4 | 2.4 | 0.8 | 3.2 | 96.1 |
| 9 | 0.8 | 0.8 | 1.6 | 97.6 | 1.0 | 0.9 | 1.9 | 97.3 | 2.8 | 0.8 | 3.6 | 95.7 |

| | RXN #19 | | | | | | | | RXN #21 | | | |
|---|---|---|---|---|---|---|---|---|---|---|---|---|
| | % | | | | RXN #20 | | | | % | | | |
| PD | HMW | | % | | % | | % | | HMW | | % | |
| Step | (70% | % | HMW | % | HMW (70% | % | HMW | % | (70% | % | HMW | % |
| # | RRT) | Dimer | (total) | Monomer | RRT) | Dimer | (total) | Monomer | RRT) | Dimer | (total) | Monomer |
| 1 | N/A | N/A | N/A | N/A | N/A | N/A | N/A | N/A | N/A | N/A | N/A | N/A |
| 2 | 0.1 | 0.9 | 0.9 | 98.4 | 0.1 | 0.8 | 0.9 | 98.3 | 0.1 | 0.8 | 0.9 | 98.3 |
| 3 | N/A | N/A | N/A | N/A | N/A | N/A | N/A | N/A | N/A | N/A | N/A | N/A |
| 4 | 0.1 | 0.9 | 0.9 | 98.4 | 0.1 | 0.8 | 0.9 | 98.3 | 0.1 | 0.9 | 1.0 | 98.3 |
| 5 | 0.4 | 0.9 | 1.3 | 98.1 | 2.3 | 0.0 | 2.4 | 96.1 | 0.8 | 0.9 | 1.7 | 97.5 |
| 6 | 0.7 | 0.8 | 1.5 | 97.7 | 3.5 | 0.7 | 4.2 | 95.0 | 1.2 | 0.8 | 2.0 | 97.2 |
| 7 | 0.7 | 0.8 | 1.5 | 97.8 | 3.5 | 0.7 | 4.2 | 95.0 | 1.1 | 0.8 | 1.9 | 97.3 |
| 8 | 0.6 | 0.8 | 1.4 | 97.8 | 3.5 | 0.7 | 4.2 | 95.0 | 0.9 | 0.8 | 1.8 | 97.4 |
| 9 | 0.3 | 0.8 | 1.1 | 98.2 | 1.5 | 0.8 | 2.3 | 96.9 | 0.5 | 0.8 | 1.4 | 97.8 |

| | RXN #22 | | | | RXN #23 | | | | RXN #24 | | | |
|---|---|---|---|---|---|---|---|---|---|---|---|---|
| | % | | | | % | | | | % | | | |
| PD | HMW | | % | | HMW | | % | | HMW | | % | |
| Step | (70% | % | HMW | % | (70% | % | HMW | % | (70% | % | HMW | % |
| # | RRT) | Dimer | (total) | Monomer | RRT) | Dimer | (total) | Monomer | RRT) | Dimer | (total) | Monomer |
| 1 | N/A | N/A | N/A | N/A | 0.0 | 0.8 | 0.9 | 98.4 | 0.0 | 0.8 | 0.9 | 98.4 |
| 2 | 0.1 | 0.9 | 0.9 | 98.4 | 0.1 | 0.8 | 0.9 | 98.4 | 0.1 | 0.8 | 0.9 | 98.4 |
| 3 | N/A | N/A | N/A | N/A | 0.1 | 0.8 | 1.0 | 98.3 | 0.1 | 0.8 | 0.9 | 98.4 |
| 4 | 0.1 | 0.9 | 1.0 | 98.3 | 0.1 | 0.8 | 1.0 | 98.3 | 0.1 | 0.9 | 0.9 | 98.4 |
| 5 | 1.4 | 0.9 | 2.3 | 97.0 | 0.4 | 0.9 | 1.3 | 98.0 | 0.3 | 0.9 | 1.2 | 98.1 |
| 6 | 2.2 | 0.8 | 3.0 | 96.3 | 0.5 | 0.8 | 1.4 | 97.9 | 0.4 | 0.8 | 1.2 | 98.1 |
| 7 | 2.3 | 0.8 | 3.1 | 96.2 | 0.4 | 0.9 | 1.3 | 97.9 | 0.3 | 0.9 | 1.2 | 98.1 |
| 8 | 2.0 | 0.8 | 2.8 | 96.5 | 0.5 | 0.9 | 1.4 | 97.9 | 0.3 | 0.9 | 1.1 | 98.1 |
| 9 | 1.4 | 0.8 | 2.2 | 97.0 | 0.3 | 0.9 | 1.2 | 98.0 | 0.2 | 0.9 | 1.1 | 98.1 |
| | RXN #25 | | | | RXN #26 | | | | RXN #27 | | | |
| | % | | | | % | | | | % | | | |
| PD | HMW | | % | | HMW | | % | | HMW | | % | |
| Step | (70% | % | HMW | % | (70% | % | HMW | % | (70% | % | HMW | % |
| # | RRT) | Dimer | (total) | Monomer | RRT) | Dimer | (total) | Monomer | RRT) | Dimer | (total) | Monomer |
| 1 | 0.0 | 0.8 | 0.9 | 98.4 | N/A | N/A | N/A | N/A | N/A | N/A | N/A | N/A |
| 2 | 0.1 | 0.9 | 0.9 | 98.4 | 0.1 | 0.8 | 0.9 | 98.5 | 0.1 | 0.8 | 0.9 | 98.5 |
| 3 | 0.1 | 0.8 | 0.9 | 98.4 | N/A | N/A | N/A | N/A | N/A | N/A | N/A | N/A |
| 4 | 0.1 | 0.8 | 0.9 | 98.4 | 0.1 | 0.8 | 0.9 | 98.4 | 0.0 | 0.8 | 0.9 | 98.5 |
| 5 | 1.3 | 0.9 | 2.2 | 97.1 | 0.4 | 0.8 | 1.2 | 98.1 | 0.2 | 0.8 | 1.1 | 98.3 |
| 6 | 1.6 | 0.8 | 2.4 | 96.8 | 0.5 | 0.8 | 1.3 | 98.0 | 0.3 | 0.8 | 1.1 | 98.2 |
| 7 | 1.7 | 0.9 | 2.6 | 96.6 | 0.5 | 0.8 | 1.3 | 98.0 | 0.3 | 0.8 | 1.2 | 98.1 |
| 8 | 1.5 | 0.9 | 2.4 | 96.9 | 0.4 | 0.8 | 1.2 | 98.1 | 0.3 | 0.8 | 1.1 | 98.2 |
| 9 | 1.3 | 0.9 | 2.2 | 97.0 | 0.4 | 0.8 | 1.2 | 98.1 | 0.2 | 0.8 | 1.1 | 98.2 |

| | RXN #28 | | | |
|---|---|---|---|---|
| | % | | | |
| PD | HMW | | | |
| Step | (70% | | % HMW | % |
| # | RRT) | % Dimer | (total) | Monomer |
| 1 | N/A | N/A | N/A | N/A |
| 2 | 0.1 | 0.8 | 0.9 | 98.5 |
| 3 | N/A | N/A | N/A | N/A |
| 4 | 0.0 | 0.8 | 0.9 | 98.5 |
| 5 | 0.9 | 0.9 | 1.7 | 97.6 |
| 6 | 1.2 | 0.8 | 2.0 | 97.3 |
| 7 | 1.2 | 0.9 | 2.1 | 97.2 |
| 8 | 1.0 | 0.9 | 1.9 | 97.4 |
| 9 | 1.1 | 0.9 | 2.0 | 97.3 |

Notes:
PD Step #: (1) pH adjusted GmAb pool (pH 9.6), (2) conjugated product pool, (3) pH adjusted conjugated product pool (pH 7.0), (4) temperature adjusted conjugated product pool (35° C.), (5) low pH conjugated product pool (pH 4.4), (6) transchelated product pool, (7) neutralized transchelated product pool (pH 7.0), (8) PD-10 product pool, (9) bulk drug substance. [symbol]
indicates data missing or illegible when filed

Several reactions which required heating and titration to pH 4.4 resulted in the formation of aggregates (HMW 70% RRT). Aggregate formation continued to occur throughout the transchelation process.

For reactions using DFO or HBED (neutral transchelating conditions), no formation of HMW species was observed.

Reactions which held the TFP-N-sucDf-Fe solution for 15 min prior to addition to GmAb (RXN #25 and #28) resulted in a relatively higher HMW content (up to 1.7%) compared to reactions that held the TFP-N-sucDf-Fe solution for 5 min (RXN #23 and #26) or 10 min (RXN #24 and #27) where HMW species reached only up to 0.5%.

Addition of the more dilute TFP-N-sucDf-Fe solution of 1.25 mg/mL resulted in relatively higher HMW formation of up to 4.4% likely due to the presence of more acetonitrile introduced to the reaction mixture.

For all reactions, the dimer content remained relatively unchanged at 0.8±0.1%.

### CAR by LC-MS

Selected BDS samples were analysed by LC-MS to determine CAR. Testing was performed using a minimum of 200 μL sample volume at approximately 2 mg/mL.

The CAR results are shown in Table 3.

TABLE 3
CAR and RCP Results of Selected Samples.

| | | | | TFP-N-sucDf-Fe | | | | | | | |
|---|---|---|---|---|---|---|---|---|---|---|---|
| | | | | Addition | | | Transchelation | | | | |
| Conjugation | | | | | Addition | | Conditions | | | Responses | |
| Reaction | pH | Mole | | Concentration | Time | TFP-N-sucDf-Fe | Transchelating | pH | Temperature | | Radiolabelling |
| # | (±0.1) | Ratio | Solvent | (mg/mL) | (min) | Manufacturer | Agent | (±0.1) | (±2° C.) | CAR | Efficiency)%) |
| 1 | 9.6 | 3 | ACN | 2.50 | 2 | ERAS | EDTA | 4.4 | 35 | 1.06 | 100 |
| 2 | 9.6 | 3 | ACN | 2.50 | 2 | ERAS | DFO | 7.0 | 22 | N/A | 95.3 |
| | | | | | | | | (±0.3) | | | |
| 3 | 9.6 | 3 | ACN | 2.50 | 2 | ERAS | HBED | 7.0 | 22 | N/A | 90 |
| | | | | | | | | (±0.3) | | | |
| 7 | 7.2 | 3 | ACN | 2.50 | 2 | ERAS | EDTA | 4.4 | 35 | 0.78 | N/A |
| 10 | 8.4 | 3 | ACN | 2.50 | 2 | ERAS | EDTA | 4.4 | 35 | 1.23 | N/A |
| 12 | 8.4 | 3 | ACN | 2.50 | 2 | ERAS | HBED | 8.4 | 22 | N/A | 64 |
| | | | | | | | | (±0.3) | | | |
| 13 | 9.6 | 3 | DMSO | 2.50 | 2 | ABX | EDTA | 4.4 | 35 | 0.96 | N/A |
| 14 | 9.6 | 3 | ACN | 5.00 | 2 | ERAS | EDTA | 4.4 | 35 | 1.10 | N/A |
| 15 | 9.6 | 3 | ACN | 1.25 | 2 | ERAS | EDTA | 4.4 | 35 | 1.13 | N/A |
| 16 | 9.6 | 3 | ACN | 2.50 | 1 | ERAS | EDTA | 4.4 | 35 | 1.36 | N/A |
| 17 | 9.6 | 3 | ACN | 2.50 | 4 | ERAS | EDTA | 4.4 | 35 | 1.09 | N/A |
| 18 | 7.2 | 3 | DMSO | 2.50 | 2 | ABX | EDTA | 4.4 | 35 | 0.59 | N/A |
| 23 | 9.6 | 3 | ACN | 2.50 | Hold | ERAS | EDTA | 4.4 | 35 | 1.04 | N/A |
| | | | | | 5 min | | | | | | |
| | | | | | Add 2 | | | | | | |
| | | | | | min | | | | | | |
| 25 | 9.6 | 3 | ACN | 2.50 | Hold | ERAS | EDTA | 4.4 | 35 | 0.92 | N/A |
| | | | | | 15 min | | | | | | |
| | | | | | Add 2 | | | | | | |
| | | | | | min | | | | | | |

Average CAR values ranged from 0.59 to 1.36.

### Radiochemical Purity

Selected BDS samples underwent radiochemical purity (RCP) testing using a minimum of 0.5 mg. RCP results are included in Table 3 above.

Radiochemical purity values ranged from 64% to 100%. An RCP of 100% was observed for the BDS obtained from RXN #1. In conditions where conjugation and transchelation (HBED transchelant) were both carried out at pH 8.4 (RXN #12), the lowest RCP of 64% was observed.

Transchelating conditions under neutral pH using DFO as the transchelant (RXN #2) resulted in a radiochemical purity of 95.3%.

### Conclusion

Several alternative conjugation and transchelating conditions were investigated in this study. Varying pH levels of 7.1, 8.4, and 9.6 were used to probe alternative conjugation strategies. Rate of linker addition, linker concentration, and hold time of linker prior to addition were also investigated. DFO and HBED were investigated as alternative transchelating agents in comparison to EDTA.

Conjugation conditions at neutral pH showed encouraging results, yielding an acceptable CAR value of 0.78. CAR values resulted in relatively little change when linker concentration was changed from 2.5 mg/mL (standard concentration) to 1.25 mg/mL and 5.0 mg/mL (CAR range of 1.06-1.13). However, an increase in aggregates was observed when the more dilute linker solution of 1.25 mg/mL was used. The hold time of linker and rate of linker addition also resulted in little change in CAR, however aggregate formation increased when TFP-N-sucDf-Fe addition hold times reached 15 min.

A high radiochemical purity of 95.3% was obtained using DFO as the transchelant under neutral pH transchelating conditions. Lower radiochemical purity was observed when HBED was used for transchelation (RXN #3: 90% and RXN #12: 64%). Transchelating conditions using EDTA resulted in the best radiochemical purity at 100%, however titration to pH 4.4 and heating were required which result in a higher % HMW, which is not suitable for larger scales.

### Example 3

Following the studies described in Examples 1 and 2, the following general procedure was developed using DFO as the free chelating ligand in the iron removal (transchelation) step. It is envisaged that other free chelating ligands, including DFO analogues and HBED, may be substituted in similar processes.

### DFO Process Overview

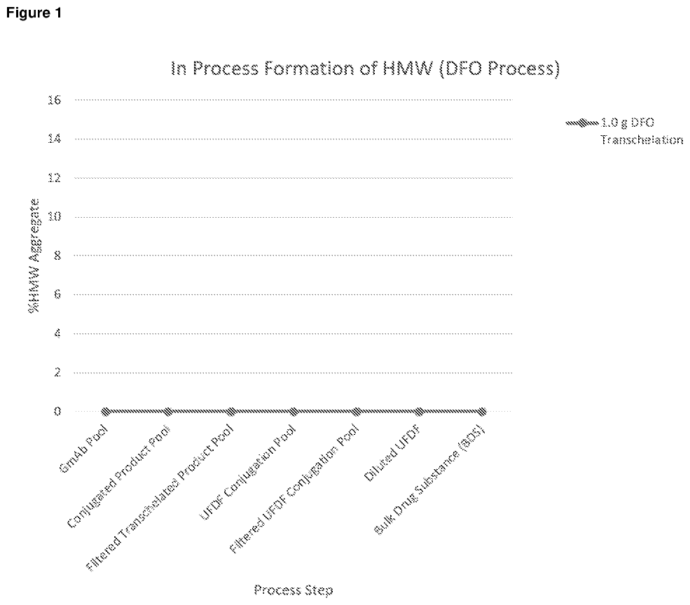

A solution of TFP-N-sucDf-Fe was prepared in ACN and added directly to the starting material GmAb Pool. The reaction was incubated at ambient temperature for 30±2 min to provide the Conjugated Product Pool. Iron was removed from GmAb-N-sucDf-Fe by the addition of free DFO. The reaction was incubated at ambient temperature to provide the transchelated product pool. The transchelated product pool was filtered using a 0.05 m² Sartobran cellulose acetate filter to provide the filtered transchelated pool. The filter was flushed with 0.9% NaCl. The filtered transchelated product pool was then subjected to a UF/DF process into 0.9% NaCl to provide UFDF Conjugation Pool. The UFDF Conjugation Pool was filtered using a 0.05 m² Sartobran cellulose acetate filter to provide the Filtered UFDF Conjugation Pool. The filter was flushed with 0.9% NaCl to ensure complete recovery of the product to yield the Filtered UFDF Conjugation Pool.
This general protocol was carried out on 1.0 g GmAb starting material. The % HMW aggregates at each stage of the process is shown in FIG. 1. FIG. 1 shows that the DFO process does not result in aggregation of the GmAb conjugates.
Results of this DFO process are summarised in Table 4.

TABLE 4

| | | | DFO-Based |
|---|---|---|---|
| Test | Test Method | Parameter | Process |
| Purity | CE-SDS (non- | % Intact IgG | 96.3% |
| | reduced) | | |
| | CE-SDS | % HC + % LC | 98.3% |
| | (reduced) | | |
| | cIEF | Main Isoform pI | 8.7 |
| | SEC-HPLC | | Monomer: |
| | | | >99% |
| | | | HMW |
| | | | Aggregate: |
| | | | not detected |
| Chelator | LC-MS | | 0.73 |
| Antibody | | | |
| Ratio (CAR) | | | |
| Radiochemical | TLC | % ⁸⁹Zr-DFO-GmAb | 100% |
| Purity | SEC-HPLC | % Monomeric | 98.5% |
| | (RAD) | ⁸⁹Zr-DFO-GmAb | |

Notes:
IgG = immunoglobulin-ie GmAb
% HC = percentage heavy chain
% LC = percentage light chain
pI = isoelectric point
TLC = thin-layer chromatography

### Example 4: Scale Up of the Process to 1.0 g

This example describes the scale up of a process similar to Example 1.

### GmAb-DFO Preparation

A solution of GmAb (5 mg/mL, 1000 mg) in 10 mM sodium phosphate, pH 7.2, 150 mM NaCl was used as the starting material.
The TFP-N-sucDf-Fe solution was prepared dissolved in ACN to a final concentration of 2.5 mg/mL.
The TFP-N-sucDf-Fe solution was added directly to the GmAb starting material at a molar ratio of 3:1 linker:mAb.
After addition of the TFP-N-sucDf-Fe solution, the reaction was incubated at room temperature with constant mixing for 30 min to generate the Conjugated Product Pool.
DFO-mesylate, for use as a transchelant to remove the Fe from the Fe complexed antibody conjugate, was prepared in water to a final concentration of 67.2 mM.
DFO-mesylate transchelant was added directly to the Conjugated Product Pool at a molar ratio of 1500:1 transchelant:mAb.
The reaction mixture was incubated at room temperature, with constant mixing, for 110 min to allow the transchelation process to go to completion and generate the Transchelated Product Pool.
The Transchelated Product Pool was filtered with a 0.05 m² 0.2 μm cellulose acetate filter prior to a UF/DF process to using a 50 kDa MWCO membrane remove unreacted TFP-N-sucDf-Fe, the DFO-meseylate-Fe complex, other small molecules and to formulate the product into final formulation buffer. This provided a UF/DF Conjugate Pool.
The UF/DF Conjugate Pool was filtered with a 0.05 m² 0.2 μm cellulose acetate filter prior to concentration adjustment to provide a Formulated Product Pool.
The Formulated Product Pool was sterile filtered using a Milipak 20 filter to provide the Bulk Drug Substance.

TABLE 5
Characterisation of GmAb-DFO prepared according to Example 4

| Test | Test Method¹ | GmAb-DFO Example 4 |
|---|---|---|
| Appearance | Visual Inspection | Clear, faint yellow, free |
| | | of particulates |
| pH | pH | 5.60 |
| Protein | Content UV (A280) | 1.97 mg/mL |
| Concentration | | |
| Purity | CE-SDS (Non- | 96.3% |
| | Reduced) | |
| | CE-SDS (Reduced) | 98.3% |
| Purity | SEC-HPLC | Monomer peak >99% |
| | | RRT(%): 100 ± 2% |
| | | HMW Aggregate 0.02% |
| | | RRT(%): ~70% |
| | cIEF | 8.723 |
| Potency & Identity | Relative Binding | 89.7% |
| | Activity³ | |
| Chelator Antibody | LC-MS | 0.74 |
| Ratio (CAR) | | |
| Residual DFO | RP-HPLC | <LOQ² |
| Radiochemical | SEC-HPLC | 98.5% |
| Purity Test | | |

Notes:
¹Tests were carried out according to general protocols described below;
²LOQ (limit of quantitation) for residual DFO is 2.21 μg/mL;
³Determined by ELISA assay.

### Example 5: Scale Up of the Process to 5.0 g

This example describes the scale up of a process similar to Example 1.

### GmAb-DFO Preparation

A solution of GmAb (5 mg/mL, 5000 mg) in 10 mM sodium phosphate, pH 7.2, 150 mM NaCl was used as the starting material.
The TFP-N-sucDf-Fe solution was prepared dissolved in ACN to a final concentration of 5.0 mg/mL.
The TFP-N-sucDf-Fe solution was added directly to the GmAb starting material at a molar ratio of 6:1 linker:mAb.
After addition of the TFP-N-sucDf-Fe solution, the reaction was incubated at room temperature with constant mixing for 30 min to generate the Conjugated Product Pool.
DFO-mesylate, for use as a transchelant to remove the Fe from the Fe complexed antibody conjugate, was prepared in water to a final concentration of 67.2 mM.
DFO-mesylate transchelant was added directly to the Conjugated Product Pool at a molar ratio of 1500:1 transchelant:mAb.
The reaction mixture was incubated at room temperature, with constant mixing, for 110 min to allow the transchelation process to go to completion and generate the Transchelated Product Pool.
The Transchelated Product Pool was subjected to a UF/DF process to using a 30 kDa MWCO membrane remove unreacted TFP-N-sucDf-Fe, the DFO-mesylate-Fe complex, other small molecules and to formulate the product into final formulation buffer. This provided a UF/DF Conjugate Pool.
The concentration of the UF/DF Conjugate Pool was adjusted to provide a Formulated Product Pool.
The Formulated Product Pool was sterile filtered using a Milipak 20 filter to provide the Bulk Drug Substance. The purity of the bulk drug substance was assessed by SEC-HPLC (according to the general protocol outlined below), which showed the following:
Monomer peak 99.4%
RRT (%): 100±2%
HMW Aggregate 0.05%
RRT (%): ˜ 70%
   The Bulk Drug substance may be further characterised by any of the techniques relied on for characterisation of the product of Example 4 and as outlined below.

### General Protocols for Analytical Tests Described in Tables 4-5

Capillary electrophoresis sodium dodecyl sulfate (CE-SDS) (non-reduced). Samples were buffer exchanged into assay buffer and mixed with iodoacetamide. Samples were heated to 70° C. and cooled before being loaded onto a 96-well plate. Samples, and appropriate controls were run using a Maurice capillary electrophoresis system.
CE-SDS (reduced). Samples were buffer exchanged into assay buffer and mixed with 2-Mercaptoethanol. Samples were heated to 70° C. and cooled before being loaded onto a 96-well plate. Samples, and appropriate controls were run using a Maurice capillary electrophoresis system.
Capillary isoelectric focussing (cIEF). Samples were diluted in water, loaded onto a 96-well plate and analysed using a Maurice capillary electrophoresis system. The relative area of acidic and basic species was determined along with the pI of the main peak.
TLC. Radiolabelled samples were spotted onto TLC paper and placed in a tank containing development buffer. Once developed, radiochromatograms were acquired and the radiolabelling efficiency was calculated as a percentage of ⁸⁹Zr bound to the GmAb-DFO.
SEC-HPLC. As described in Example 2.
SEC-HPLC (RAD). Radiolabelled samples were diluted, as appropriate, with mobile phase. Samples were analysed using a Yarra SEC-3000 column. Data were acquired using both a UV and radioactivity detector. Radiochemical purity was determined by comparing the peak area of monomeric ⁸⁹Zr-DFO-GmAb with the total observable peak area.
Liquid chromatography-mass spectrometry (LC-MS). LC-MS operated under standard methods, with a column capable of separating conjugated antibody from unconjugated antibody.
Relative Binding Activity. May be measured by any means known in the art. In some embodiments, relative binding activity may be determined by an ELISA assay or any other suitable immunological assay.

1. A process for preparing a conjugate of a desferrioxamine chelating ligand linked with a protein, the process comprising exposing an iron complexed conjugate comprising the desferrioxamine chelating ligand complexed to iron linked with the protein, to a free chelating ligand selected from desferrioxamine, a desferrioxamine analogue and HBED, or a combination thereof, at substantially neutral pH to thereby remove iron from the desferrioxamine chelating ligand of the iron chelated conjugate and provide the conjugate of the desferrioxamine chelating ligand linked with the protein that is substantially free of chelated iron.

2. The process of claim 1, wherein the protein is a protein targeting agent.

3. The process of claim 1 or 2, wherein the protein is an antibody.

4. The process of any one of claims 1-3, wherein the protein is girentuximab.

5. The process of any one of claims 1-4, wherein the desferrioxamine chelating ligand is selected from DFO, DFO*, DFOSq, DFONCS, DFO*Sq and DFO*NCS.

6. The process of any one of claims 1-5, wherein the peptide or protein is linked to the desferrioxamine chelating ligand through a covalent linker selected from a covalent bond and succinyl.

7. The process of any one of claims 1-6, wherein the step of exposing the iron complexed conjugate with the free chelating agent is conducted at ambient temperature.

8. The process of claim 7, wherein the ambient temperature is about 20° C. to about 25° C.

9. The process of any one of claims 1-8, wherein the substantially neutral pH is physiological pH.

10. The process of any one of claims 1-8, wherein the pH is from about 7 to about 8.

11. The process of claim 10, wherein the pH is about 7.2 or about 7.4.

12. The process of any one of claims 1-11, wherein the conjugate is exposed to the free chelating ligand in a solvent comprising water.

13. The process of any one of claims 1-12, wherein the desferrioxamine analogue is selected from DFO*, DFOSq, DFONCS, DFO*Sq and DFO*NCS.

14. The process of any one of claims 1-13, wherein the iron complexed conjugate is exposed to a molar excess of the free chelating agent.

15. The process of claim 14, wherein a molar ratio of the iron complexed conjugate to the free chelating agent is from about 1:50 to about 1:5000.

16. The process of any one of claims 1-15, further comprising, prior to the exposing step, conjugating an iron chelated desferrioxamine chelating ligand with a protein to provide a conjugate of the protein linked with the desferrioxamine chelating ligand complexed to iron.

17. A process of preparing a conjugate of a desferrioxamine chelating ligand linked with a protein, the process comprising combining at substantially neutral pH:
a first composition comprising an iron chelated desferrioxamine chelating ligand and a polar organic solvent and
a second composition comprising a protein,
to thereby form an iron complexed conjugate of the desferrioxamine chelating ligand complexed to iron linked with the protein.

18. The process of claim 17, wherein the polar organic solvent comprises acetonitrile (ACN), dimethylsulfoxide (DMSO) or a combination thereof.

19. The process of claim 17 or 18, wherein the process comprises combining a molar excess of the iron chelated desferrioxamine chelating ligand relative to the protein.

20. The process of claim 19, wherein the molar excess is at least about 2 molar equivalents of the iron chelated desferrioxamine chelating ligand relative to the protein.

21. The process of any one of claims 17-20, further comprising exposing the iron complexed conjugate comprising the desferrioxamine chelating ligand complexed to iron linked with the protein, to a free chelating ligand selected from desferrioxamine, a desferrioxamine analogue and HBED, or a combination thereof, at substantially neutral pH to thereby remove iron from the desferrioxamine chelating ligand of the iron chelated conjugate and provide the conjugate of the desferrioxamine chelating ligand linked with the protein that is substantially free of chelated iron.

22. A process of preparing a conjugate of a desferrioxamine chelating ligand linked with a protein, the process comprising:
conjugating an iron chelated desferrioxamine chelating ligand with a protein to provide an iron complexed conjugate of the desferrioxamine chelating ligand complexed to iron linked with the protein; and
exposing the iron complexed conjugate comprising the desferrioxamine chelating ligand complexed to iron linked with the protein, to a free chelating ligand selected from desferrioxamine, a desferrioxamine analogue and HBED, or a combination thereof, at substantially neutral pH to thereby remove iron from the desferrioxamine chelating ligand of the iron chelated conjugate and provide the conjugate of the desferrioxamine chelating ligand linked with the protein that is substantially free of chelated iron.

23. A protein conjugate produced by the process of any one of claims 1-22.
